SEQUENCE LISTING (10 sequences)

SEQ ID NO: 1 - DNA, 2480 bp, Artificial sequence
  transcriptional unit comprising promoter, coding sequence for transcription factor of SEQ ID NO2 and terminator elements
aggatattaaagtatgtattcatcattaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgagctcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagaactagtggatcccccgggccctgcaggctcgagctagtttgagatatccccgttatggtactggggttgcatataacccattccttggttgtatgctccctgttggcccatcccttgtgcagctgagctacttgctcccacatgaccaagggcatcctttttaattgagccatcgctagattttgcagttaacttgctatcaccctccatctctctgtacttctgcaggtacaccttgaggggttcaatgtagtcttcaaaccccagcgtggccatggcccacagcagatcgtcgccattgatggtcttccgcttctccctctggcacttgtcactcgcttcgctagtgatgaaggagatgaactcggagacgcactcctgcacggtctccttagcgtccttggcgatcttcccgttagccgggatggtcttcccgttagccgggatggccttcttcatgatgcgactgatgttggcgatgggcaggaacctgtcctgctccctgacgctgccaccgcctccgcctcccctggggctcccgctctcgtggctcccgccgccgccgccagggctcgccggagcttccgccatggtctacctacaaaaaagctccgcacgaggctgcatttgtcacaaatcatgaaaagaaaaactaccgatgaacaatgctgagggattcaaattctacccacaaaaagaagaaagaaagatctagcacatctaagcctgacgaagcagcagaaatatataaaaatataaaccatagtgcccttttcccctcttcctgatcttgtttagcatggcggaaattttaaaccccccatcatctcccccaacaacggcggatcgcagatctacatccgagagccccattccccgcgagatccgggccggatccacgccggcgagagccccagccgcgagatcccgcccctcccgcgcaccgatctgggcgcgcacgaagccgcctctcgcccacccaaactaccaaggccaaagatcgagaccgagacggaaaaaaaaaacggagaaagaaagaggagaggggcggggtggttaccggcggcggcggagggggaggggggaggagctcgtcgtccggcagcgaggggggaggaggtggtggtggtggtggtggtagggttggggggatgggaggagaggggggggtatgtatatagtggcgatggggggcgtttctttggaagcggagggagggccggcctcgtcgctggctcgcgatcctcctcgcgtttccggcccccacgacccggacccacctgctgttttttctttttcttttttttctttctttttttttttttggctgcgagacgtgcggtgcgtgcggacaactcacggtgatagtgggggggtgtggagactattgtccagttggctggactggggtgggttgggttgggttgggttgggctgggcttgctatggatcgtggatagcactttgggctttaggactttaggggttgtttttgtaaatgttttgagtctaagtttatcttttatttttactagaaaaaatacccatgcgctgcaacgggggaaagctattttaatcttattattgttcattgtgagaattcgcctgaatatatatttttctcaaaaattatgtcaaattagcatatgggtttttttaaagatatttcttatacaaatccctctgtatttacaaaagcaaacgaacttaaaacccgactcaaatacagatatgcatttccaaaagcgaataaacttaaaaaccaattcatacaaaaatgacgtatcaaagtaccgacaaaaacatcctcaatttttataatagtagaaaagagtaaatttcactttgggccaccttttattaccgatattttactttataccaccttttaactgatgttttcacttttgaccaggtaatcttacctttgttttattttggactatcccgactctcttctcaagcatatgaatgacctcgag

SEQ ID NO: 2 - protein, 185 aa, Zea mays
MAEAPASPGGGGGSHESGSPRGGGGGGSVREQDRFLPIANISRIMKKAIPANGKTIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAQGMGQQGAYNQGMGYMQPQYHNGDISN

SEQ ID NO: 3 - protein, 178 aa, Zea mays
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAEGMGQQGAYNQGMGYMQPQYHNGDISN

SEQ ID NO: 4 - DNA, 537 bp, Zea mays
atggcggaagctccggcgagccctggcggcggcggcgggagccacgagagcgggagccccaggggaggcggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctccgagttcatctccttcatcactagcgaagcgagtgacaagtgccagagggagaagcggaagaccatcaatggcgacgatctgctgtgggccatggccacgctggggtttgaagactacattgaacccctcaaggtgtacctacagaagtacagagagatggagggtgatagcaagttaactgctaaatctagcgatggctcgattaaaaaggatgctcttggtcatgtgggagcaagtagctcagctgcagaagggatgggccaacagggagcatacaaccaaggaatgggttatatgcaacctcagtaccataacggggatatctcaaactaa

SEQ ID NO: 5 - DNA, 522 bp, Glycine max
atgtcggatgcgccaccgagcccgactcatgagagtgggggcgagcagagcccgcgcggttcgtcgtccggcgcgagggagcaggaccggtacctcccgattgccaacatcagccgcattatgaagaaggctctgcctcccaacggcaagattgcaaaggatgccaaagacaccatgcaggaatgcgtttctgagttcatcagcttcattaccagcgaggcgagtgagaaatgccagaaggagaagagaaagacaatcaatggagacgatttgctatgggccatggccactttaggatttgaagactacatagagccgcttaaggtgtacctggctaggtacagagaggcggagggtgacactaaaggatctgctagaagtggtgatggatctgctacaccagatcaagttggccttgcaggtcaaaattctcagcttgttcatcagggttcgctgaactatattggtttgcaggtgcaaccacaacatctggttatgccttcaatgcaaagccatgaatag

SEQ ID NO: 6 - protein, 173 aa, Glycine max
MSDAPPSPTHESGGEQSPRGSSSGAREQDRYLPIANISRIMKKALPPNGKIAKDAKDTMQECVSEFISFITSEASEKCQKEKRKTINGDDLLWAMATLGFEDYIEPLKVYLARYREAEGDTKGSARSGDGSATPDQVGLAGQNSQLVHQGSLNYIGLQVQPQHLVMPSMQSHE

SEQ ID NO: 7 - protein, 141 aa, Arabidopsis thaliana
MADTPSSPAGDGGESGGSVREQDRYLPIANISRIMKKALPPNGKIGKDAKDTVQECVSEFISFITSEASDKCQKEKRKTVNGDDLLWAMATLGFEDYLEPLKIYLARYRELEGDNKGSGKSGDGSNRDAGGGVSGEEMPSW

SEQ ID NO: 8 - protein, 101 aa, Artificial sequence
  protein consensus sequence
REQDRYLPIANISRIMKKALPXNGKXXXXXXXIAKDAKXTXQECVSEFISFITSEASXKCQXEKRKTINGDDLLWAMATLGFEDYIEPLKVYLXXYREXEG

SEQ ID NO: 9 - protein, 55 aa, Artificial sequence
  consensus protein sequence
DSKLTAKSSDGSIKKDALGHVGASSSAAXGMGQQGAYNQGMGYMQPQYHNGDISN

SEQ ID NO: 10 - protein, 8 aa, Artificial sequence
  consensus protein sequence
MXXXPXSP

(19) United States
(12) Patent Application Publication
(10) Pub. No.: US 2005/0022266 A1
(43) Pub. Date: Jan. 27, 2005

(54) Yield-improved transgenic plants

(76) Inventors: Jingrui Wu, Chesterfield, MO (US); Garrett J. Lee, St. Louis, MO (US); Thomas R. Adams, North Stonington, CT (US); Zhidong Xie, Maryland Heights, MO (US); Stanton B. Dotson, Chesterfield, MO (US); Donald E. Nelson, Stonington, CT (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/678,588

(22) Filed: Oct. 2, 2003

Related U.S. Application Data
(60) Provisional application No. 60/415,758, filed on Oct. 2, 2002.
(60) Provisional application No. 60/463,787, filed on Apr. 18, 2003.
(60) Provisional application No. 60/425,157, filed on Nov. 8, 2002.

(51) Int. Cl. A01H 1/00; C12N 15/82

(52) U.S. Cl. 800/289; 435/468

(57) ABSTRACT

Disclosed herein are transgenic plants having a recombinant DNA construct which expresses a Hap3 transcription factor which provides enhanced resistance and/or tolerance to water deficit. More specifically the DNA constructs comprise a polynucleotide which encodes at least a functional part of a transcription factor that is a CCAAT-box DNA binding subunit or a homologous transcription factor.

### RELATED APPLICATIONS

This application claims priority to provisional applications No. 60/415,758; 60/425,157 and 60/463,787, the disclosures of all of which are incorporated herein by reference.

### INCORPORATION OF SEQUENCE LISTING

The sequence listing is identical to the sequence listing submitted in provisional application No. 60/463,787, for Water-Deficit-tolerant Transgenic Plants (docket No. 38-21(52578)B, where the computer readable form was in the file named “ZMCAAT-2.5T25.txt” which is 14 kilobytes (measured in MS-Windows), was created on Apr. 17, 2003, was submitted on a 3.5″ diskette, and is incorporated herein by reference.

### FIELD OF THE INVENTION

Disclosed herein are DNA useful for producing transgenic plants and methods of using such DNA for producing transgenic plants and seed.

### BACKGROUND OF THE INVENTION

One of the goals of plant genetic engineering is to produce plants with agronomically, horticulturally or economically important traits including tolerance to any of a variety of environmental stresses. The yield from a plant is influenced by environmental factors including water availability, exposure to cold or heat, availability of nutrients such a phosphorus and nitrogen, plant density and the like. A plant's response to such environmental stress can be influenced by internal genetic mechanisms.

Considering the complexity of water use in land plants, especially during conditions that produce water deficit, relatively few genes specifically associated with this aspect of physiology have been identified. It would be of benefit to the art to increase the number and variety of genes involved in regulating water use in plants, more particularly, in crop plants such as corn, soybean, cotton, wheat, canola and the like which are commonly grown in locations subject to water deficit. Thus, a particular object of this invention is to identify protein transcription factors which are beneficial to the plant when produced during water deficit.

Transcription factors have been investigated for improving plant properties and traits in transgenic plants. Li et al. in Nucleic Acid Res. 20(5), 1087-1091 (1992) discloses a Zea mays gene which encodes a transcription factor described as a CCAAT-box DNA binding protein subunit B. Edwards et al., (Plant Physiol 117:1015-1022, 1998) demonstrated that multiple genes exist for each of the HAP2, 3, 5 subunits in Arabidopsis, providing the potential for multiple alternative forms of HAP complexes in plants. Homologs are disclosed by Harada et al. in U.S. Pat. No. 6,235,975 which are alleged to be useful for modulating embryo development in transgenic plants.

Many crop plants are transgenic and comprise genes that impart herbicide and/or insect resistance traits. Incorporation of additional transgenic genes for enhancing yield in crop plants presents a challenge of using DNA constructs of increased complexity.

### SUMMARY OF THE INVENTION

We have discovered that over expression of certain genes encoding Hap3 transcription factors having a CCAAT-box DNA binding protein impart to plants a significant resistance and/or tolerance to water deficit. The present invention uses genes which encode at least a water-deficit-tolerance-imparting functional part of a Hap3 transcription factor which is useful in transgenic plants for enhancing yield when the plants are subjected to water deficit. Thus, one aspect of this invention provides methods for providing transgenic plants with an enhanced resistance and/or tolerance to water deficit. More particularly the method comprises transforming plants with a recombinant DNA construct which confers resistance to and/or tolerance to water deficit. Another aspect of the invention provides transgenic seed for growing a plant which is resistant to water deficit as compared to wild type wherein the genome of said seed comprises a recombinant DNA construct which expresses a Hap3 transcription factor of this invention or a water-deficit tolerance-imparting homolog. Still another aspect of this invention relates to plants grown from such transgenic seed. Transformed plants with tolerance and/or resistance to water deficit should inherently provide enhanced yield as compared to wild type plants which are retarded by or succumb to water deficit.

### BRIEF DESCRIPTION OF THE DRAWINGS

FIG. 1 is an amino acid sequence alignment.

### DETAILED DESCRIPTION OF THE INVENTION

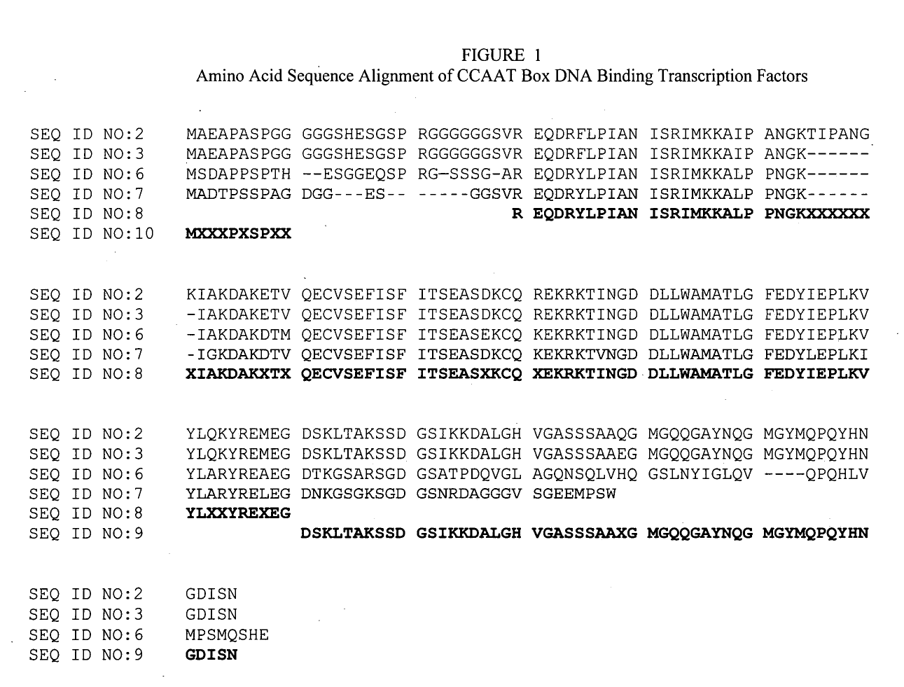

The Hap3 transcription factors of this invention, which confer water deficit tolerance and/or resistance when constitutively expressed in a transgenic plant, are in a class known as CCAAT box binding DNA binding proteins. Zea mays Hap3 transcription factors have amino acid sequences of SEQ ID NO: 2 and 3; a homologous soybean (Glycine max) Hap3 transcription factor has an amino sequence of SEQ ID NO:6; an Arabidopsis thaliana Hap3 transcription factor has an amino acid sequence of SEQ ID NO:7. The amino acid sequence of the transcription factors of this invention, i.e. protein sequences of SEQ ID NO: 2, 3, 6 and 7, were aligned as illustrated in FIG. 1 to identify regions of common sequence. SEQ ID NO:8 is an artificial consensus sequence for a region of about 100 amino acid residues common to all four transcription factors. SEQ ID NO:9 is an artificial consensus sequence for a region of about 50 amino acid residues common to a terminus of the Zea mays transcription factors. SEQ ID NO:10 is an artificial consensus sequence for a region of eight amino acid residues common to a terminus of all four transcription factors. Aspects of this invention include transgenic plants having exogenous DNA which expresses homologous genes encoding transcription factors which are functionally equivalent to the transcription factors demonstrated to provide water deficit tolerance. Such homologous genes will encode transcription factors which have a core amino acid sequence which is the same as or at least 90% identical to consensus sequence of SEQ ID NO:8 and have a terminal amino acid sequence which is the same as or at least 90% identical to either or both of the consensus sequence of SEQ ID NO:9 and SEQ ID NO:10.

SEQ ID NO: 1 provides the DNA sequence for an exogenous transcriptional unit comprising promoter elements, DNA encoding a Zea mays transcription factor and terminator elements.

SEQ ID NO:2 provides the amino acid sequence of a transcription factor encoded by the DNA encoding a Zea mays transcription factor within the exogenous transcriptional unit of SEQ ID NO:1. The transcription factor is described as a CCAAT-box DNA binding protein subunit B.

SEQ ID NO:3 provides the amino acid sequence for a Zea mays transcription factor that is homologous to the transcription factor with the amino acid sequence of SEQ ID NO:2. The transcription factor of SEQ ID NO:3 was originally disclosed by Li et al. in Nucleic Acid Res. 20(5), 1087-1091 (1992).

SEQ ID NO:4 provides the DNA sequence of a Zea mays gene that encodes the transcription factor of SEQ ID NO:3.

SEQ ID NO:5 provides. DNA sequence of a Glycine max gene that encodes a transcription factor that is homologous to the Zea mays transcription factors of SEQ ID NO:2 and 3.

SEQ ID NO:6 provides the amino acid sequence for the a Glycine max transcription factor that is encoded by the DNA of SEQ ID NO:5.

SEQ ID NO:7 provides the amino acid sequence of an Arabidopsis thaliana transcription factor that is homologous to the Zea mays transcription factors of SEQ ID NO:2 and 3.

SEQ ID NO:8 is an artificial consensus amino acid sequence of a common core region of amino acids of the transcription factors of SEQ ID NO:2, 3, 6 and 7.

SEQ ID NO:9 is an artificial consensus amino acid sequence of a common terminus region of the Zea mays transcription factors of SEQ ID NO:2 and 3.

SEQ ID NO:10 is an artificial consensus amino acid sequence of a common terminus region of eight amino acids of the transcription factors of SEQ ID NO:2, 3, 6 and 7.

As used herein “water deficit” means a deprivation of water sufficient to at least retard growth and development in a wild type plant. Extreme water deficit is sufficient to cause wilt and plant death. Irrigated crops can experience water deficit in extreme heat when the transpiration rate is greater than water uptake. In comparative assays a “water deficit” condition is conveniently characterized by water potential in a plant tissue of less than −0.7 megapascals (MPa), e.g. −0.8 Mpa. Water potential in corn is conveniently measured by clamping a leaf segment in a pressurizable container so that a cut cross section of leaf is open to atmospheric pressure. Gauge pressure (above atmospheric pressure) on the contained leaf section is increased until water begins to exude from the atmospheric-pressure-exposed cross section; the gauge pressure at incipient water exudation is reported as negative water potential in the plant tissue, e.g. 7 bars of gauge pressure is reported as −0.7 MPa water potential. Water deficit can be induced by withholding water from plants for sufficient time that wild type plants are deleteriously affected, e.g. as manifested by reduced yield, stunted growth, retarded development, death or the like. The plants of this invention show a remarkable risibility after periods of water deficit that are adverse to wild type plants.

As used herein “yield” of a crop plant means the production of a crop, e.g. shelled corn kernels or soybean or cotton fiber, per unit of production area, e.g. in bushels per acre or metric tons per hectare, often reported on a moisture adjusted basis, e.g. corn is typically reported at 15.5% moisture. Moreover a bushel of corn is defined by law in the State of Iowa as 56 pounds by weight, a useful conversion factor for corn yield is: 100 bushels per acre is equivalent to 6.272 metric tons per hectare. Other measurements for yield are in common practice.

As used herein a “transgenic” organism, e.g. plant or seed, is one whose genome has been altered by the incorporation of a trait-conferring, recombinant DNA, e.g. exogenous DNA or additional copies of native DNA, e.g. by transformation or by breeding with a transformed plant. Thus, transgenic plants include progeny plants of an original plant derived from a transformation process including progeny of breeding transgenic plants with wild type plants or other transgenic plants. The enhancement of a desired trait can be measured by comparing the trait property in a transgenic plant which has recombinant DNA conferring the trait to the trait level in a progenitor plant. As used herein “progenitor plant” refers to a plant of essentially the same genotype as a transgenic plant but lacking the specific trait-conferring, recombinant DNA that characterizes the transgenic plant. Such a progenitor plant that lacks that recombinant DNA can be a natural, wild-type plant, an elite, non-transgenic plant, or a transgenic plant without the specific trait-conferring, recombinant DNA that characterizes the transgenic plant. The progenitor plant lacking the specific, trait-conferring recombinant DNA can be a sibling of a transgenic plant having the specific, trait-conferring recombinant DNA. Such a progenitor sibling plant may comprise other recombinant DNA.

Crop plants of interest in the present invention include, but are not limited to, soybean (including the variety known as Glycine max), cotton, canola (also known as rape), corn (also known as maize and Zea mays), wheat, sunflower, sorghum, alfalfa, barley, millet, rice, tobacco, fruit and vegetable crops and turfgrasses.

As used herein an “herbicide resistance” trait is a characteristic of a transgenic plant that is resistant to dosages of an herbicide that is typically lethal to a progenitor plant. Such herbicide resistance can arise from a natural mutation or more typically from incorporation of recombinant DNA that confers herbicide resistance. Herbicides for which resistance is useful in a plant include glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides and gluphosinate herbicides. To illustrate the that production of transgenic plants with herbicide resistance is a capability of those of ordinary skill in the art reference is made to U.S. patent application publications 2003/0106096A1 and 2002/0112260A1 and U.S. Pat. Nos. 5,034,322; 6,107,549 and 6,376,754, all of which are incorporated herein by reference.

As used herein an “pest resistance” trait is a characteristic of a transgenic plant is resistant to attack from a plant pest such as a virus, a nematode, a larval insect or an adult insect that typically is capable of inflicting crop yield loss in a progenitor plant. Such pest resistance can arise from a natural mutation or more typically from incorporation of recombinant DNA that confers pest resistance. For insect resistance, such recombinant DNA can, for example, encode an insect lethal protein such as a delta endotoxin of Bacillus thuringiensis bacteria or be transcribed to a dsRNA targeted for suppression of an essential gene in the insect. To illustrate that the production of transgenic plants with pest resistance is a capability of those of ordinary skill in the art reference is made to U.S. Pat. Nos. 5,250,515 and 5,880,275 which disclose plants expressing an endotoxin of Bacillus thuringiensis bacteria, to U.S. Pat. No. 6,506,599 which discloses control of invertebrates which feed on transgenic plants which express dsRNA for suppressing a target gene in the invertebrate, to U.S. Pat. No. 5,986,175 which discloses the control of viral pests by transgenic plants which express viral replicase, and to U.S. Patent Application Publication 2003/0150017 A1 which discloses control of pests by a transgenic plant which express a dsRNA targeted to suppressing a gene in the pest, all of which are incorporated herein by reference.

Protein and Polypeptide Molecules—As used herein “protein” means a polypeptide of combined amino acids including a natural protein or polypeptide fragment of a natural protein or a modified natural protein or a synthetic protein, or a peptide having a protein function. Proteins produced and used by the transgenic plants of this invention are whole proteins or at least a sufficient portion of an entire protein to impart the relevant biological activity of the protein, e.g. a crop improvement trait. To illustrate, reference is made to Hap3 proteins which are effective in conferring water deficit tolerance in transgenic plants, e.g. when constitutively expressed. The Hap3 proteins are transcription factors having a CCAAT-box DNA binding domain and include the Zea mays transcription factors of SEQ ID NO: 2 and 3, the Glycine max transcription factor of SEQ IS NO:6 and the Arabidopsis thaliana transcription factor of SEQ ID NO:7 or a functionally-equivalent homologous transcription factor. Such functionally-equivalent homologous transcription factor can be defined by the consensus amino acid sequences that characterize these transcription factors. With reference to FIG. 1 the defining consensus sequences are the central amino acid region consensus sequence of SEQ ID NO:8 and at least one of the terminus amino acid consensus sequences of SEQ ID NO:9 and SEQ ID NO:10.

Aside from similarity in function homologs of proteins or DNA can be described as molecules having a sequence, e.g. amino acid or nucleotide sequence, which shares identity to a reference sequence. For simplicity, DNA homologs are defined by optimally aligning the nucleotide sequence of a putative DNA homolog with a defined nucleotide sequence and determining identical nucleotide elements over a window of the defined sequence. Similarly, protein homologs of a consensus amino acid sequence is defined by optimally aligning the amino acid sequence of a putative protein homolog with a defined amino acid sequence and determining the identical amino acid elements over a window of the defined sequence. Optimal alignment can be effected manually but more preferably with the assistance of a homology-based search algorithms such as those commonly known and referred to as BLAST, FASTA, and Smith-Waterman.

For other than consensus amino acid sequences protein homologs are defined by optimally aligning the amino acid sequence of a putative protein homolog with a defined amino acid sequence and determining the conservatively substituted amino acid elements over a window of the defined sequence. Conservatively substituting amino acids are (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; (4) neutral nonpolar (hydrophobic) amino acids such as alanine, leucine, isoleucine, valine, proline, phenylalanine, tryptophan, and methionine; (5) amino acids having aliphatic side chains such as glycine, alanine, valine, leucine, and isoleucine; (6) amino acids having aliphatic-hydroxyl side chains such as serine and threonine; (7) amino acids having amide-containing side chains such as asparagine and glutamine; (8) amino acids having aromatic side chains such as phenylalanine, tyrosine, and tryptophan; (9) amino acids having basic side chains such as lysine, arginine, and histidine; (10) amino acids having sulfur-containing side chains such as cysteine and methionine. To account for insertions and deletions, mutations, variations in the C and/or N terminal regions of proteins and non-conservative substitutions, protein homologs are defined as being at least 80% identical.

Recombinant DNA Constructs—The present invention contemplates the use of polynucleotides which encode a protein effective for imparting resistance and/or tolerance to water deficit in plants. Such polynucleotides are assembled in recombinant DNA constructs using methods known to those of ordinary skill in the art. A useful technology for building DNA constructs and vectors for transformation is the GATEWAY™ cloning technology (available from Invitrogen Life Technologies, Carlsbad, Calif.) uses the site specific recombinase LR cloning reaction of the Integrase/att system from bacterophage lambda vector construction, instead of restriction endonucleases and ligases. The LR cloning reaction is disclosed in U.S. Pat. Nos. 5,888,732 and 6,277,608, U.S. Patent Application Publications 2001283529, 2001282319 and 20020007051, all of which are incorporated herein by reference. The GATEWAY™ Cloning Technology Instruction Manual which is also supplied by Invitrogen also provides concise directions for routine cloning of any desired RNA into a vector comprising operable plant expression elements.

Recombinant DNA constructs used for transforming plant will comprise DNA cells for conferring a trait along with other commonly used DNA elements As is well known in the art such constructs typically also comprise a promoter and other regulatory elements, 3′ untranslated regions (such as polyadenylation sites), transit or signal peptides and marker genes elements as desired. For instance, see U.S. Pat. Nos. 5,858,742 and 5,322,938 which disclose versions of the constitutive promoter derived from cauliflower mosaic virus (CaMV35S), U.S. Pat. No. 6,437,217 which discloses a maize RS81 promoter, U.S. Pat. No. 5,641,876 which discloses a rice actin promoter, U.S. Pat. No. 6,426,446 which discloses a maize RS324 promoter, U.S. Pat. No. 6,429,362 which discloses a maize PR-1 promoter, U.S. Pat. No. 6,232,526 which discloses a maize A3 promoter, U.S. Pat. No. 6,177,611 which discloses constitutive maize promoters, U.S. Pat. No. 6,433,252 which discloses a maize L3 oleosin promoter, U.S. Pat. No. 6,429,357 which discloses a rice actin 2 promoter and intron, U.S. Pat. No. 5,837,848 which discloses a root specific promoter, U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, U.S. Pat. No. 6,175,060 which discloses phosphorus deficiency inducible promoters, U.S. Patent Application Publication 2002/0192813A1 which discloses 5′, 3′ and intron elements useful in the design of effective plant expression vectors, U.S. patent application Ser. No. 09/078,972 which discloses a coixin promoter, U.S. patent application Ser. No. 09/757,089 which discloses a maize chloroplast aldolase promoter, all of which are incorporated herein by reference.

In many aspects of the invention it is preferred that the promoter element in the DNA construct should be capable of causing sufficient expression to result in the production of an effective amount of the transcription factor in water deficit conditions. Such promoters can be identified and isolated from the regulatory region of plant genes which are over expressed in water deficit conditions. Alternatively, such promoters can be exogenous constitutive promoters. Another class of useful promoters are water-deficit-inducible promoters, e.g. promoters which are derived from the 5′ regulatory region of genes identified as a heat shock protein 17.5 gene (HSP 17.5), an HVA22 gene (HVA22), and a cinnamic acid 4-hydroxylase (CA4H) gene (CA4H) of Zea maize; such water-deficit-inducible promoters are disclosed in U.S. provisional application Ser. No. 60/435,987, filed Dec. 20, 2002, incorporated herein by reference. Another water-deficit-inducible promoter is derived from the rab-17 promoter as disclosed by Vilardell et al., Plant Molecular Biology, 17(5):985-993, 1990.

In general it is preferred to introduce heterologous DNA randomly, i.e. at a non-specific location, in the plant genome. In special cases it may be useful to target heterologous DNA insertion in order to achieve site specific integration, e.g. to replace an existing gene in the genome. In some other cases it may be useful to target a heterologous DNA integration into the genome at a predetermined site from which it is known that gene expression occurs. Several site specific recombination systems exist which are known to function implants include cre-10× as disclosed in U.S. Pat. No. 4,959,317 and FLP-FRT as disclosed in U.S. Pat. No. 5,527,695, both incorporated herein by reference.

In practice DNA is introduced into only a small percentage of target cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Transformation Methods and Transgenic Plants—Methods and compositions for transforming plants by introducing a recombinant DNA construct into a plant genome in the practice of this invention can include any of the well-known and demonstrated methods. Preferred methods of plant transformation are microprojectile bombardment as illustrated in U.S. Pat. Nos. 5,015,580; 5,550,318; 5,538,880; 6,160,208; 6,399,861 and 6,403,865 and Agrobacterium-mediated transformation as illustrated in U.S. Pat. Nos. 5,635,055; 5,824,877; 5,591,616; 5,981,840 and 6,384,301, all of which are incorporated herein by reference. See also U.S. application Ser. No. 09/823,676, incorporated herein by reference, for a description of vectors, transformation methods, and production of transformed Arabidopsis thaliana plants where transcription factors are constitutively expressed by a CaMV35S promoter.

Transformation methods of this invention to provide plants with enhanced environmental stress tolerance are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Those cells which are capable of proliferating as callus also are recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. Nos. 6,194,636 and 6,232,526 and U.S. patent application Ser. No. 09/757,089, which are incorporated herein by reference.

The seeds of this invention can be harvested from fertile transgenic plants and be used to grow progeny generations of transformed plants of this invention including hybrid plants line comprising the DNA construct expressing a transcription factor which provides the benefits of resistance and/or tolerance to water deficit.

Having now generally described the invention, the same will be more readily understood through reference to the following example which is provided by way of illustration, and is not intended to be limiting of the present invention, unless specified. These examples illustrates the use of polynucleotides encoding a water-deficit tolerance-imparting transcription factor to provide various transgenic plants exhibiting enhanced tolerance for and/or resistance to growing conditions of water deficit.

Breeding of Transgenic Plants

In addition to direct transformation of a plant with a recombinant DNA construct, transgenic plants can be prepared by crossing a first plant having a recombinant DNA construct with a second plant lacking the construct. For example, recombinant DNA can be introduced into a plant line that is amenable to transformation to produce a transgenic plant which can be crossed with a second plant line to introgress the recombinant DNA into the second plant line.

In one aspect of the invention a transgenic plant with recombinant DNA conferring a crop improvement trait is crossed with a transgenic plant having recombinant DNA conferring herbicide and/or pest resistance to produce progeny plants having recombinant DNA that confers both the crop improvement trait and the herbicide and/or pest resistance trait. Preferably, in such breeding for combining traits the transgenic plant donating the crop improvement trait is a female line and the transgenic plant donating the herbicide and/or pest resistance trait is a male line. The progeny of this cross will segregate such that some of the plant will carry the DNA for both parental traits and some will carry DNA for one parental trait; such plants can be identified by markers associated with parental recombinant DNA Progeny plants carrying DNA for both parental traits can be crossed back into the female parent line multiple times, e.g. usually 6 to 8 generations, to produce a progeny plant with substantially the same genotype as one original transgenic parental line but for the recombinant DNA of the other transgenic parental line.

In yet another aspect of the invention hybrid transgenic seed, e.g. a hybrid transgenic corn seed, is produced by crossing a female transgenic corn line containing recombinant DNA conferring a crop improvement trait with a male transgenic corn line containing recombinant DNA conferring herbicide and/or pest resistance. In a preferred aspect of this invention hybrid transgenic corn seed is produced by crossing a female transgenic corn line with recombinant DNA conferring both a crop improvement trait and herbicide resistance with a male transgenic corn line with recombinant DNA conferring both herbicide resistance and pest resistance.

### EXAMPLE 1

This example illustrates aspects of the invention through the production of transgenic corn plants and seed with recombinant DNA that confers water-deficit tolerance.

Transgenic corn was transformed with a recombinant DNA construct having the nucleotide sequence of SEQ ID NO:1 and which comprises a rice actin 1 constitutive promoter and a rice actin 1 intron operably linked an corn gene which encodes a Hap3 transcription factor with the amino acid sequence of SEQ ID NO:2 followed by a Tr7 3′ terminator. The construct further comprised a CaMV 35 S promoter operably linked to an nptII marker gene. Twenty transgenic events in corn were selected as having either 1 or 2 copies of the construct and no oriV origin of replication from the vector. Eleven of the twenty events survived to fertile plants which produced seed. Seed producing plants were analyzed to verify the presence of the exogenous DNA encoding the transcription factor and accumulation of the transcription factor. Six of the transgenic events were used in a water deficit assay.

Pre-germinated seedlings of transgenic plants (progeny of a heterozygous transgenic plant which inherited the exogenous transcription factor DNA construct) and wild type plants (progeny of a heterozygous transgenic plant which did not inherit the exogenous transcription factor DNA construct) were planted in 5 inch pots containing 330 grams of soil. The plants were well watered for one week then allowed to dry for 4 days. An equal number (32) of transgenic and wild type plants were selected based on matched height and the selected plants were mixed in eight flats. Four flats were designated as “wet” meaning they would be well watered and four flats were designated as “dry” meaning they would be subject to water deficit. All pots were brought to the weight of the heaviest pot by adding water. The pots were weighed daily until the average pot weight dropped to between 600 to 700 grams, whereupon a water deficit assay was started by measuring plant heights and resuming watering for pots in “wet” flats while continuing to withhold water for pots in “dry” flats.

The pots in the “wet” flats were fully-watered daily. The pots in the “dry” flats were weighed daily to determine a water deficit treatment. If the average “dry” flat pot weight was greater than 500 grams, no water was added; if the average pot weight was between 365 and 500 grams, 35 grams of water was added to each pot; and, if the average pot weight was less than 365 grams, a determined amount of water was added to bring the average pot weight to 400 grams. The water deficit treatment was continued until the pots in the “dry” flats have had an average pot weight below 500 grams for 8 days. The height of all plants was measured on the 9th day and averages are reported in Table 1.

TABLE 1
Average Change in Plant Height (cm) through first water deficit

| Treatment | Transgenic | Wild Type | Ratio | Significance |
|---|---|---|---|---|
| Full water | 46.3 cm | 45.5 cm | 1.02 | 0.03 |
| Water deficit | 20.5 cm | 19.7 cm | 1.04 | 0.015 |

On the 9^{th} day full watering was resumed for the “dry” flat pots for 3 days when heights were again measured. See tables 2 and 3 for changes in average plant height for the eight water-deficited plants which were recovered during the 3-day recovery period. Table 2 shows the average incremental change in plant height for the recovered plants which occurred during the 3-day recovery period. Table 3 show the total average change in plant height for the recovered plants which occurred through the combined water-deficit and recovery periods.

TABLE 2
Average Change in Recovered Plant Height During 3-day Recovery Period

| Treatment | Transgenic | Wild Type | Ratio | Significance |
|---|---|---|---|---|
| Water recovered | 13.2 cm | 13.2 cm | 1.0 | 0.74 |

TABLE 3
Average Change in Recovered Plant Height Through Deficit and Recovery

| Treatment | Transgenic | Wild Type | Ratio | Significance |
|---|---|---|---|---|
| Water deficit | 33.8 cm | 32.9 cm | 1.03 | 0.03 |

Recovered plants were subjected to a second round of water deficit as described above. After 9 days full water was resumed for 7 days. After two days of full water the twice water-deficited plants began to show signs of recovery from a wilted state. In some cases recovery took 5-6 days and some plants never recovered. On average the recovered transgenic plants were significantly greener and healthier than recovered wild type plants which were more wilted and yellow (indicating senescence).

### EXAMPLE 2

This example further illustrates the aspect of this invention relating to transgenic corn. Progeny seed of the transgenic corn produced in Example 1 was planted in a field trial to evaluate its water deficit tolerance as compared to the negative sibling (wild type). The plants were grown in a well irrigated field in Kansas. Water was withheld from half of the planting during the late vegetative stage. The experimental evidence showed that under water deficit conditions transgenic corn plants expressing the Hap3 transcription factor of SEQ ID NO:2 were healthier than the wild type and exhibited the following phenotypes:
(a) likely to have a higher chlorophyll index, e.g. >42 in transgenic plants as compared to <40 in wild type,
(b) likely to produce more photosynthate,
(c) likely to have cooler leaf temperature, and
(d) likely to maintain higher stomatal conductance.

### EXAMPLE 3

This example illustrates the invention through the preparation of transgenic seed and plants with a crop improvement trait, e.g. water-deficit tolerant soybean.

Transgenic soybean was transformed with a recombinant DNA construct comprising a CaMV 35S constitutive promoter operably linked to an Arabidopsis thaliana gene which encodes the Hap3 transcription factor having the amino acid sequence of SEQ ID NO:7 followed by a terminator element. In a water deficit assay Transgenic soybean plants exhibited enhanced resistance to water deficit, i.e. less wilting, as compared to wild type soybean plants. In addition In particular, transgenic plants wilted less, had a higher chlorophyll content, had a higher relative water content, had a higher photosynthesis rate, than their gene negative segregants and parental control plants.

### EXAMPLE 4

This example illustrates the invention through the preparation of transgenic seed and plants with a crop improvement trait, e.g. water-deficit tolerant soybean.

Transgenic soybean was transformed with a recombinant DNA construct comprising a CaMV 35S constitutive promoter operably linked to an endogenous soybean gene having the nucleotide sequence of SEQ ID NO:5 which encodes the native Hap3 transcription factor having the amino acid sequence of SEQ ID NO:6 followed by a terminator element. In a water deficit assay where water was withheld after saturating potted plants at the V1 stage until the soil reached 10% of capacity (50% for well watered control), transgenic soybean plants exhibited enhanced resistance to water deficit as compared to wild type soybean plants. In particular, transgenic plants wilted less, had a higher chlorophyll content, had a higher relative water content, had a higher photosynthesis rate, than their gene negative segregants and parental control plants.

### EXAMPLE 5

This example illustrates aspects of the invention through the preparation of transgenic seed and plants with a crop improvement trait and herbicide and insect resistance traits.

Transgenic maize is transformed with recombinant DNA constructs substantially as disclosed in Example 1 except that the selective marker is an EPSPS gene that confers resistance to glyphosate herbicide. The transgenic plants produce seed which can be used to grow water-deficit-tolerant progeny plant which can be bred with transgenic plants having pest resistance to provide progeny plants with stacked engineering traits.

Seed from plants with water deficit tolerance and glyphosate herbicide tolerance are used as female plants in breeding with a pollen from transgenic plants with insect resistance, e.g. maize line MON863 available from Monsanto Company, St. Louis, Mo., which was contains recombinant DNA expressing the cry3Bb1 gene encoding a Coleopteran-specific insecticidal protein from Bacillus thuringiensis (subsp. kumamotoensis) to control infestation with corn root worm (CRW; Diabrotica sp). Segregating progeny plants are selected for all three traits, i.e. water deficit tolerance, herbicide tolerance and insect resistance. Selected plants are back crossed for 6 generations with the water deficit tolerant line. By such breeding the insect resistance trait is introgressed into the transgenic line with water deficit tolerance and glyphosate herbicide tolerance.

### EXAMPLE 6

This example illustrates another aspect of the invention through the preparation of transgenic seed and plants with a crop improvement trait and herbicide tolerance and insect resistance traits.

Transgenic maize is transformed with recombinant DNA constructs substantially as disclosed in Example 1 except that the selective marker is an bar gene that confers tolerance to glufosinate herbicide. Seed from water deficit-tolerant, glufosinate herbicide-tolerant plants were used a female plants in breeding with a pollen from a glyphosate herbicide-tolerant, insect-resistant transgenic corn plants, e.g. maize line MON802 available from Monsanto Company, St. Louis, Mo. and which has recombinant genes encoding the CrylAb protein from Bacillus thuringiensis and the 5-enolpyruvylshikimate-3-phosphate synthase (EPSPS) from A. tumefaciens strain CP4. Segregating progeny plants are selected for water deficit tolerance by screening with glufosinate herbicide and insect resistance by screening with glyphosate herbicide.

1. A method for improving yield in a crop exposed to water deficit by providing a transgenic seed for said crop wherein said transgenic seed has a recombinant DNA construct expressing a gene which encodes
(a) a Hap3 protein having at least 80% identity to an amino acid sequence of SEQ ID NO:2, SEQ ID NO:3, SEQ ID NO:6 or SEQ ID NO:7 determined by an amino acid window of said sequence; or
(b) a Hap3 protein having an amino acid sequence identical to a consensus amino acid sequence of SEQ ID NO:8, SEQ ID NO:9 or SEQ ID NO:10.

2. A method of claim 1 wherein said crop is corn, soybean, canola, wheat, rice, cotton or grass.

3. A method of claim 1 wherein said recombinant DNA construct comprises said gene operably linked for transcription to a water-deficit-inducible promoter.

4. A method of claim 3 wherein said water-deficit-inducible promoter is a Rab-17, Hva22, Ca4H, Hsp17.5 promoter.

5. A method for improving water-deficit survivability of a plant comprising introducing into the genome of said plant a recombinant DNA construct expressing a gene which encodes
(a) a Hap3 protein having at least 80% identity to an amino acid sequence of SEQ ID NO:2, SEQ ID NO:3, SEQ ID NO:6 or SEQ ID NO:7 determined by an amino acid window of said sequence; or
(b) a Hap3 protein having an amino acid sequence identical to a consensus amino acid sequence of SEQ ID NO:8, SEQ ID NO:9 or SEQ ID NO:10.

6. Water-deficit-tolerant, transgenic, hybrid maize comprising a recombinant DNA construct expressing a gene which encodes
(a) a Hap3 protein having at least 80% identity to an amino acid sequence of SEQ ID NO:2, SEQ ID NO:3, SEQ ID NO:6 or SEQ ID NO:7 determined by an amino acid window of said sequence; or
(b) a Hap3 protein having an amino acid sequence identical to a consensus amino acid sequence of SEQ ID NO:8, SEQ ID NO:9 or SEQ ID NO:10.

7. Water-deficit-tolerant, transgenic soybean comprising a recombinant DNA construct expressing a gene which encodes
(a) a protein having at least 80% identity to an amino acid sequence of SEQ ID NO:2, SEQ ID NO:3, SEQ ID NO:6 or SEQ ID NO:7 determined by an amino acid window of said sequence; or
(b) a Hap3 protein having an amino acid sequence identical to a consensus amino acid sequence of SEQ ID NO:8, SEQ ID NO:9 or SEQ ID NO:10.

8. A transgenic seed having in its genome a recombinant DNA construct which expresses a gene which encodes a Hap3 protein having a consensus amino acid sequence of SEQ ID NO:10 and a gene imparting herbicide resistance.

9. A seed of claim 8 wherein said gene imparting herbicide resistance provides resistance to an herbicide selected from the group consisting of a glyphosate herbicide, a phosphinothricin herbicide, an oxynil herbicide, an imidazolinone herbicide, a dinitroaniline herbicide, a pyridine herbicide, a sulfonylurea herbicide, a bialaphos herbicide, a sulfonamide herbicide and a gluphosinate herbicide.

10. A seed of claim 8 wherein said genome further comprises a recombinant DNA construct which expresses a gene encoding an insecticidal protein.

11. A seed of claim 10 wherein said insecticidal protein is a delta endotoxin from Bacillus thurengienisus.

12. A seed of claim 8 wherein said genome further comprises a recombinant DNA construct which is transcribed to RNA which forms gene silencing dsRNA targeted to a crop pest.

13. A plant produced from a seed of claim 8.
